(19) United States
(12) Patent Application Publication
(10) Pub. No.: US 2023/0340521 A1
(43) Pub. Date: Oct. 26, 2023

(54) NEMATODE RESISTANCE IN PLANTS

(71) Applicant: CHRISTIAN-ALBRECHTS-UNIVERSITÄT ZU KIEL, Kiel (DE)

(72) Inventors: Christian Jung, Kiel (DE); Avneesh Kumar, Kiel (DE)

(21) Appl. No.: 17/925,550

(22) PCT Filed: May 19, 2021
(86) PCT No.: PCT/EP2021/063385

§ 371 Date: Nov. 15, 2022

(30) Foreign Application Priority Data
May 19, 2020 (EP) 20175552.7

(51) Int. Cl. C12N 15/82; C12Q 1/6895

(52) U.S. Cl. CPC C12N 15/8285; C12Q 1/6895; C12Q 2600/13; C12Q 2600/158

(57) ABSTRACT

The invention provides a gene, herein termed Hs4, which encodes a protein, also referred to as Hs4, which upon presence in plants confers resistance against plant parasitic nematodes, especially against the beet cyst nematode Heterodera schachtii Schmidt. Specifically, the invention provides the use of plants containing the Hs4 gene for cultivation in the presence of nematodes, in soil of which the presence of nematodes is unknown and in soil that is free from nematodes

The present invention relates to a novel resistance gene which upon expression in plants confers resistance against nematodes, especially of the genus Heterodera, e.g. against the beet cyst nematode Heterodera schachtii Schmidt. For the purpose of the present invention, the novel resistance gene, and respectively the protein encoded by the novel resistance gene, is termed Hs4. The invention provides plants which carry the Hs4 gene, a process for breeding plants using the Hs4 gene as a marker for identifying plants during the breeding process that carry the Hs4 gene, a process for generating plants that carry the Hs4 gene by genetic manipulation to introduce or modify the Hs4 gene into plants, and to a nucleic acid construct comprising or consisting of the coding sequence of the Hs4 gene under the control of a promoter that is active in plants, which promoter can be a constitutive promoter and which promoter preferably is a promoter that is induced upon nematode infection, e.g. the Hs1 beet cyst nematode resistance gene promotor.

### STATE OF THE ART

Cai et al., Science vol. 275, 832-834 (1997) describe the cloning of a gene encoding a 282 amino acid protein that in sugar beet provides resistance against Heterodera schachtii.
Schulte et al., Mol Gen Genomics (2006) describe the generation of a complete physical map of a wild beet translocation site in sugar beet and further mention that the resistance gene described in Cai et al., Science (1997), did not confer complete nematode resistance after transformation into sugar beet.
Heller et al., Theor Appl Genet 92: 991-997 (1996) describe linkage analysis using restriction fragment length polymorphism (RFLP) analysis for localizing nematode resistance genes in sugar beets and for identification of markers linked to the resistance.
EP 3567111 A1 describes three proteins which upon expression in beets confer nematode resistance. The proteins have a length of 1084 amino acids, of 1429 amino acids and of 965 amino acids, respectively.
WO 2014/127835 A1 describes two resistance genes encoding proteins having approx. 929 amino acids for nematode resistance in beets.
Savitsky, H. Can. J. Genet. Cytol. 20:177-186 (1978) describes crossing Beta vulgaris and Beta procumbens resulting in a translocation of part of chromosome 1 of Beta procumbens onto the chromosome 9 of Beta vulgaris.
Fauser et al., The Plant Journal 79, 348-359 (2014) describes a method and a plasmid for integration of DNA sections into a plant genome by homologous recombination, making use of CRISPR/Cas methods, especially in a Cas9 expression system plasmid called pChimera.
Jager, doctoral thesis entitled “Hybrid assembly of whole genome shotgun sequences of two sugar beet (Beta vulgaris L.) translocation lines carrying the beet cyst nematode resistance gene Hs1-2 and functional analysis of candidate genes” (2012) focuses on an ORF702, located on a BAC contig, as a potential nematode resistance gene. This genome section turned out not to be the resistance gene, as no significant differences in nematode development were found in over-expressing and control plants. The search for sequences that display homology to known genes involved in plant pathogen resistance did not lead to identification of a novel nematode resistance gene.

### OBJECT OF THE INVENTION

It is an object of the invention to provide an alternative resistance gene that confers resistance against the beet cyst nematode into plants, especially for highly specific identification of the resistance gene in plants, for introducing the resistance gene into plants by genetic manipulation, and to provide a plant breeding process in which plants bearing the resistance gene are identified using the resistance gene as a probe. Preferably, the resistance gene shall provide high resistance against nematodes, especially in Amaranthaceae.

### DESCRIPTION OF THE INVENTION

The invention achieves the object by the features of the claims, and especially provides a gene, herein termed Hs4, which encodes a protein, also referred to as Hs4, having an amino acid sequence having a homology of at least 50%, e.g. of at least 60%, of at least 70%, preferably of at least 80%, preferably of at least 85%, more preferably of at least 90% or of at least 95%, most preferably of at least 98% or 99% to M E A V F W I I L L N F V I Y G A E H L G R V E E I R T L Y L I H D R P A W Y Q F V T S A F C H Y N W N H L C N N L F F L Y I F G K L V E E E V G G F Y L W Y Y Y I L T A V G S N L V S W S L L P R S G S S A G A S G A V F G L F A I S F S V K L L L R D R C P D N K E D W R R F L E V I I L G H F V L Q R M M E A L H G S N A M V N A N G V I D P A L V P W V N H I A H L A G A V V G M S L V I I P H D I R R R L S V N N C L P P (SEQ ID NO: 1), and/or to MEAIILLNFVIYGAEHLGRVEEIRTLYLIHDRPAWYQFVTSAFCHYNWNHLCNNLFFL YIFGKLVEEEVGGFYLWYYYILTAVGSNLVSWSLLPRSGSSAGASGAVFGLFAIS FSVKLLLRDRCPDNKEDWRRFLEVIILGHFVLQRMMEALHGSNAMVNANGVIDPAL VPWVNHIAHLAGAVVGMSLVIIPHDIRRRLSVNNCLPP (SEQ ID NO: 17), especially of the amino acid sequence of SEQ ID NO: 1 or of SEQ ID NO: 17, which upon presence in plants confers resistance against plant parasitic nematodes, especially against the beet cyst nematode Heterodera schachtii Schmidt. Specifically, the invention provides the use of plants containing the Hs4 gene for cultivation in the presence of nematodes, in soil of which the presence of nematodes is unknown and in soil that is free from nematodes, which nematodes preferably are Heterodera schachtii. In an embodiment, the invention provides for plants that carry a Hs4 encoding gene as the only resistance gene against nematodes, as it has been found that the Hs4 encoding gene encodes a protein that by itself, e.g. without additional nematode resistance genes, confers resistance against nematodes and protects against nematodes. It was found that the Hs4 having amino acid sequence of SEQ ID NO: 17 is shorter than the Hs4 of SEQ ID NO: 1 by three amino acids, both conferring resistance against nematodes when expressed in plants. Generally herein, the resistance gene Hs4 refers to a nucleic acid sequence encoding a protein having a homology of at least 50%, e.g. of at least 60%, of at least 70%, preferably of at least 80%, preferably of at least 85%, more preferably of at least 90% or of at least 95%, most preferably of at least 98% or 99% to one or both of SEQ ID NO: 1 and SEQ ID NO: 17. Herein, reference to Hs4 includes both SEQ ID NO: 1 and the variant Hs4_1 having SEQ ID NO: 17.

The protein HS4 can be expressed from a cDNA having a DNA sequence having a homology of at least 80%, preferably of at least 85%, more preferably of at least 90% or of at least 95%, most preferably of at least 98% or 99% to SEQ ID NO: 2, or from a sequence containing exons and introns, e.g. a sequence having a homology of at least 80%, preferably of at least 85%, more preferably of at least 90% or of at least 95%, most preferably of at least 98% or 99% to SEQ ID NO: 3, or having the sequence of SEQ ID NO: 3.

Plants carrying the Hs4 gene show only little or no yield penalty, i.e. less or no deterioration of yield in production, when cultivated in nematode-free soil, preferably also in nematode-infested soil, compared to wild-type plants without the Hs4 gene cultivated in nematode-free soil. Especially preferred are plants that for nematode resistance carry only the Hs4 gene, e.g. after genetic modification or as part of a small translocation from the P. procumbens which can be obtained after crossing different translocation lines or by mutagenesis.

Preferably, a plant containing the Hs4 gene contains e.g. only a DNA portion containing the Hs4 gene encoding section and regulatory genetic elements for its expression, which DNA portion consists of at maximum 3 kbp, more preferably at maximum 2.7 kbp, or more preferred of at maximum 1 kbp. The DNA portion containing the Hs4 gene encoding section and regulatory genetic elements for its expression can be introduced into the plant as a small translocation, e.g. from P. procumbens, or by genetic manipulation. Most preferably, the plant in addition to its original DNA only contains the Hs4 gene comprising or consisting of regulatory genetic elements functionally linked to a coding sequence encoding the Hs4 gene product, wherein the regulatory genetic elements preferably include a promoter that is induced by presence of nematodes, optionally at least one enhancer in 5′ of the coding sequence or in 5′ of the promoter. Accordingly, the plant, e.g. in comparison to the genome of the nematode-susceptible cultivar, essentially contains no additional DNA in excess of the Hs4 gene, which comprises or consists of a coding sequence for the Hs4 protein and functionally linked regulatory genetic elements. Preferably, the plant containing the Hs4 gene can contain an insert comprising the Hs4 gene, which insert has a size of at maximum 100,000 bp, more preferably of at maximum 10,000 bp. The size of the insert can e.g. be determined in comparison to the genome of the plant prior to the integration of the insert comprising the Hs4 gene.

A preferred promoter that is functionally linked to the nucleic acid sequence encoding the protein having a homology of at least 50%, e.g. of at least 60%, of at least 70%, preferably of at least 80%, preferably of at least 85%, more preferably of at least 90% or of at least 95%, most preferably of at least 98% or 99% to SEQ ID NO: 1 or SEQ ID NO: 17, or of SEQ ID NO: 1 or SEQ ID NO: 17, is a promoter that is inducible by presence of nematodes. A preferred promoter that is inducible by presence of nematodes is the promoter of SEQ ID NO: 4, which is also termed Hs1_prom (comprised of nucleotides No. 9 . . . 1477 of SEQ ID NO: 15). As an alternative, the promoter having a homology, e.g. of at least 50%, preferably of at least 80%, more preferably of at least 85%, more preferably of at least 90% or of at least 95%, most preferably of at least 98% or 99% to SEQ ID NO: 4, or comprising or consisting of SEQ ID NO: 4, can be comprised in an expression cassette encoding another protein, e.g. a protein conferring resistance against plant parasitic nematodes, and the expression cassette comprising the promoter can be present in a plant, e.g. by genetic manipulation or by a translocation event.

Preferably, the plant is a member of Brassicales, e.g. Brassicaceae, of Amaranthaceae, of Brassicaceae, of Solanaceae, or of Poaceae, e.g. Graminae, especially barley, wheat and rice, as well as banana, sugar cane. Although the invention is not limited to a specific cultivar, the plant preferably is a cultivar of one of the aforementioned genera. Optionally, from the plants according to the invention there are excluded Patellifolia procumbens, formerly known as Beta procumbens, and/or Patellifolia patellaris.

The plant may be a hybrid or a doubled haploid plant, which do not occur in nature. The present invention shows that the Hs4 encoding gene provides resistance in plants by providing nematode resistance to Brassicales, exemplified by Arabidopsis thaliana, by introducing the Hs4 encoding gene, and by showing that inactivation of the Hs4 encoding gene in an originally nematode resistant plant results in susceptibility to nematodes.

Analysis of the Hs4 gene product revealed that upon presence in a plant the Hs4 gene expresses a protease. Currently it is assumed that the efficacy of the Hs4 gene to provide resistance against nematodes is based on a detrimental effect of the protease activity on nematodes or on the functioning of the nematode feeding sites. Accordingly, the invention also relates to plants expressing Hs4, wherein Hs4 has protease activity and confers resistance against plant parasitic nematodes. Currently, the Hs4 protein is believed to encode a rhomboid-like protease, which is predicted to be bound to the endoplasmic reticulum of the plant cells. As the mechanism of activity of the Hs4 protein is not limited to specific plants, presence of the Hs4 encoding gene can provide nematode resistance in other plants, e.g. without limitation to a specific plant family or plant species. An advantage of Hs4 being a rhomboid-like protease is that these preserve their functional activity also against their substrates from different organisms and that they do not require cofactors, e.g. for catalyzing intramembrane enzymatic reactions.

As the present invention provides the amino acid sequence of the causative gene product for nematode resistance, analysis of plant material for determining the presence of a nucleic acid sequence encoding the causative gene product has become feasible by nucleic acid analytical methods, or by immunological methods that are specific for the Hs4 gene product. The identification of the Hs4 gene therefore allows the use of in vitro methods for determining the nematode resistance of a plant, and therefore facilitates the identification of plants that carry the nematode resistance in plant breeding, e.g. reducing the necessity for determining nematode resistance in nematode infection experiments.

The resistance gene Hs4 can be used for identifying plants that carry a nucleic acid sequence encoding the Hs4 gene, e.g. using nucleic acid sequences that hybridize to a nucleic acid sequence encoding a protein having a homology of at least 80%, preferably of at least 85%, more preferably of at least 90% or of at least 95%, most preferably of at least 98% or 99% to SEQ ID NO: 1. Processes for identifying the presence of nucleic acid sequences are generally known, e.g. processes using hybridization of nucleic acid probes that are specific, e.g. in PCR, Southern hybridization, or DNA sequencing.

The use of the resistance gene Hs4 in a process for analysis of plants in respect of the presence of a nucleic acid encoding the Hs4 gene is useful e.g. in processes for plant breeding for identifying plants that have acquired the Hs4 gene. Processes for plant breeding can comprise or consist of crossing plants and selecting offspring plants that carry the Hs4 gene.

Further, the invention relates to a process for genetically manipulating plants by integrating a nucleic acid sequence encoding the Hs4 gene into the plant genome, and also relates to the genetically manipulated plants, e.g. obtainable by genetic manipulation. According to an embodiment of the invention, genetically manipulated plants contain an insert which essentially consists of the Hs4 gene including regulatory genetic elements for expression of a protein of SEQ ID NO: 1 or SEQ ID NO: 17, with essentially no additional DNA sections. E.g. the insert preferably contains or consists of the coding sequence arranged under the control of a promoter, which preferably is a promoter that is induced by presence of nematodes, e.g. the promoter of SEQ ID NO: 4, and a terminator.

Generally, genetically manipulated plants according to the invention contain an insert comprising the Hs4 gene which is integrated into the plant genome by genetic manipulation. Methods for genetic manipulation of plants for integrating DNA into plant genomes are generally known, e.g. using Agrobacterium containing a plasmid which comprises a DNA section encoding the Hs4 gene for transformation of plants using inoculation of the plants with the Agrobacterium. Alternatively or additionally, integration of DNA sections into the plant genome can be obtained by genome editing, e.g. CRISPR-Cas based methods.

Methods for genetic manipulation of native protease genes with >80% DNA sequence homology to the Hs4 gene by genome editing, e.g. CRISPR-Cas based methods. Single or oligonucleotide mutations can be induced in Hs4 homologs of crop plants, preferably from the plant families Amaranthaceae, Brassicaceae, Poaceae, Solanaceae, whose Hs4 homologs originally are without a function as a nematode resistance gene, in order to alter their function into a nematode resistance gene, which preferably encodes a protein having a homology of at least 50%, e.g. of at least 60%, of at least 70%, preferably of at least 80%, preferably of at least 85%, more preferably of at least 90% or of at least 95%, most preferably of at least 98% or 99%, or consisting of, SEQ ID NO: 1, which protein preferably is a protease.

The Hs4 gene of the invention has the advantage of being of plant origin, and to encode a protein that does not significantly impair yield of cultivated plants, e.g. of sugar beets.

The figures show in

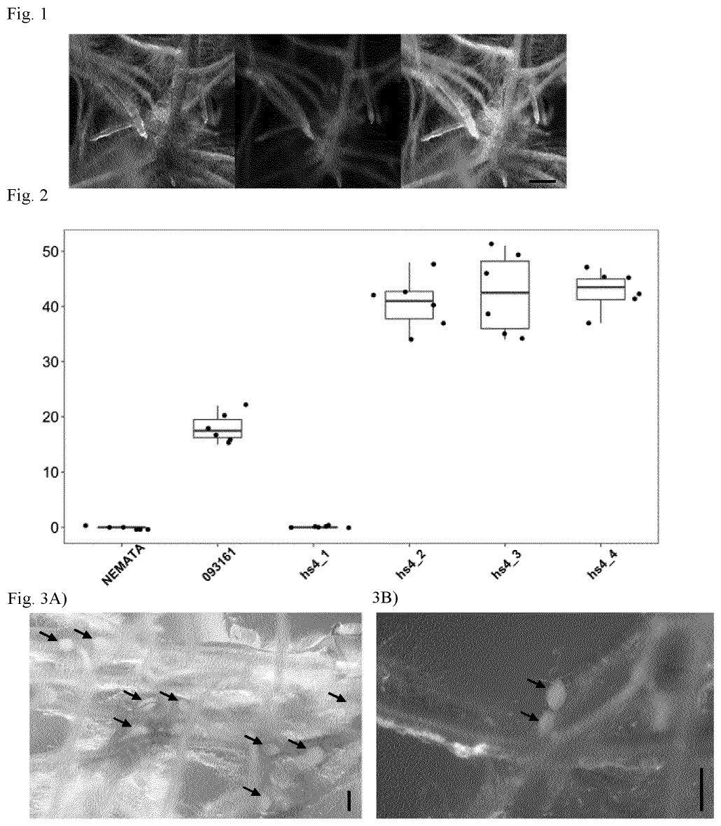

FIG. 1 fluorescence microscopic images of 3-week old root clones from NEMATA expressing GFP and carrying the CRIPR-Cas9 cassette for knock-out of the Hs4 gene,

FIG. 2 a box plot showing that expression of the resistance gene of the invention correlates with reduction of infection by nematodes,

FIG. 3 shows microscopic images of hairy roots infected with nematodes, A) of susceptible sugar beet line 93161 as a positive control, B) of Nemata roots in which the Hs4 gene has been knocked out by CRISPR-Cas technology turning a resistant into a susceptible root,

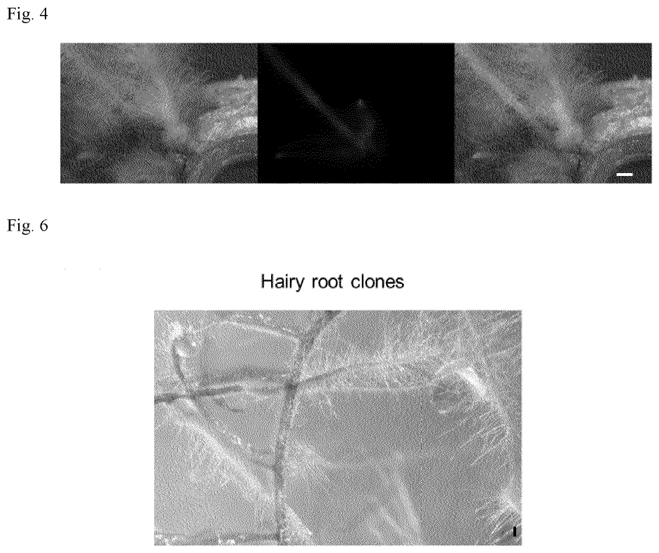

FIG. 4 fluorescence microscopic images of 12-d-old hairy roots genetically manipulated to express the RFP gene as a reporter gene and the Hs4-overexpression cassette,

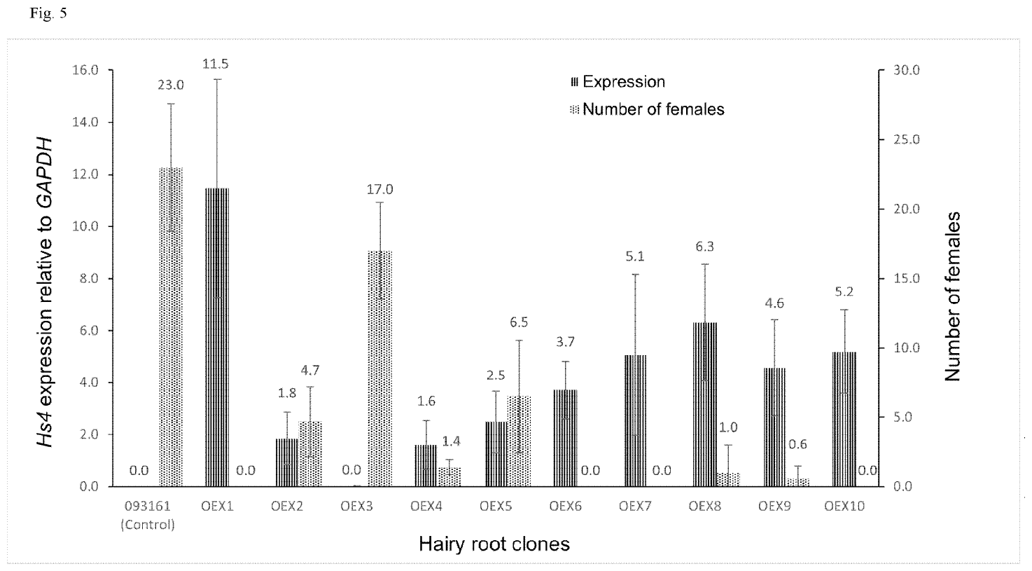

FIG. 5 a graphic presentation of the expression levels of the Hs4 resistance gene (left column) in relation to expression of the housekeeping gene GAPDH, and number of female nematodes, and

FIG. 6 a microscopic image of hairy roots of a clone expressing the Hs4 gene in the presence of infecting nematodes.

In the microscopic images, the bar is 1000 μm.

The invention is now described in greater detail by way of examples. In the examples, the resistance gene product is also referred to as the Hs4 gene. Nematodes were Heterodera schachtii Schmidt that were propagated under non-sterile conditions on susceptible sugar beet plants. Fully developed brown cysts were harvested from the roots onto 50 m sieves. A 3 mM ZnCl₂ solution was used to stimulate the hatching of juveniles in the dark. Nematodes were examined under a binocular microscope. Only suspensions with >90% mobile nematodes were taken as inoculum. For in vitro tests, nematodes were surface-sterilized by soaking them in 0.05% HgCl₂ solution for 30 s, and were then washed four times with sterile water and resuspended in 0.2% (w/v) Gelrite (Duchefa Biochemie BV, Haarlem, the Netherlands). In all, 250 sterile nematodes were used to inoculate the hairy roots. For glasshouse resistance tests, plants were grown in 20 ml tubes filled with sterile sand (grain size 0.1-1.5 mm), sterilized at 80° C. for 3 h. Six hundred freshly hatched second-stage juveniles (J2 larvae) were added to each plant with a syringe. At 4 weeks after infection, plants were harvested and washed, and roots were examined under a binocular microscope. In the in vitro tests, root clones were inoculated with 250 nematodes and roots were examined for resistance after 4 weeks. For RNA isolation, root samples were collected 3, 6, 9 and 12 d post-inoculation (dpi), with three biological replicates per sample.

A comparison of the Hs4 protein sequence to the sequences analysed in Jager, quoted above, showed that the Hs4 gene was not among the BAC sequences analysed in Jager.

### Example 1: Analysis of the Presence of the Hs4 Gene in a Plant

As an example for analytical methods for determining the presence of specific nucleic acid sequences encoding a protein having a homology of at least 80% to SEQ ID NO: 1, the DNA from leaf samples or root samples from sugar beets was purified. The DNA was analysed by PCR, using the primer of SEQ ID NO: 5 and SEQ ID NO: 6, which between them specifically hybridize to a section of the natural Hs4 gene, comprising the first exon and part of the 5′UTR of SEQ ID NO: 3. Generally, primer pairs that are specific for amplifying a portion of a known DNA sequence, can be designed using generally available computer programmes on the Hs4 gene sequence. Amplification conditions were annealing at 58° C., polymerase synthesis at 72° C. for 60 s, denaturing at 94° C. for 30 s, for 35 cycles. The amplification product had a size of 194 bp.

### Example 2: Generating a Plant that Contains the Hs4 Gene by Genetic Manipulation

Transfer of the resistance gene Hs4 into plants can be made using a Ti plasmid and Agrobacterium tumefaciens or a Ri plasmid and Agrobacterium rhizogenes, wherein the plasmid comprises an expression cassette encoding the Hs4 gene product.

Alternatively, a nucleic acid sequence encoding a protein of at least 50% homology, preferably of at least 80% homology, more preferably of at least 89% homology to SEQ ID NO: 1 can be integrated into the genome of a plant using a CRISPR-Cas based method.

Another method for introducing the nematode resistance gene Hs4 into a plant which originally is devoid of the functional Hs4 gene, especially into sugar beets, more preferably into sugar beet varieties, comprises the steps of crossing the plant with a Hs4 gene containing plant and from offspring of the crossing selecting those that contain the Hs4 gene in combination with a desired combination of traits of the plant which originally is devoid of the functional Hs4 gene. The presence of the Hs4 gene can be determined by analysing plant material according to Example 1.

### Example 3: Nematode Resistance is Caused by the Hs4 Gene

The sugar beet (Beta vulgaris) variety Nemata, which is known as nematode resistant, was genetically manipulated to inactivate the Hs4 gene specifically. The inactivation of the Hs4 gene directly resulted in nematode susceptibility of the plants.

In short, target sites for integration were identified manually. As target sites, SEQ ID NO: 7 and SEQ ID NO: 8 were used, which target the exon region of the sugar beet. These target sites were cloned into the pChimera vector (described by Fauser et al., 2014, kindly provided by Prof. Dr. Holger Puchta to the Plant Breeding Institute, CAU Kiel). A single guide RNA having SEQ ID NO: 9 and SEQ ID NO: 10 were cloned into the vector p201G (described by Jacobs et al. 2015, obtainable from Addgene) separately and transformed into Agrobacterium rhizogenes. Leaf stalks of the sugar beet were inoculated with the transformed A. rhizogenes, and obtained hairy roots were kept on Gamborg B5 medium for two weeks. Hairy roots were tested for mutation in or around target sites by PCR using SEQ ID NO: 11 and SEQ ID NO: 12 as primers and were confirmed by Sanger sequencing. Hairy roots were then inoculated with 250 of J2 Heterodera schachtii nematodes, and after four weeks of subsequent cultivation in the dark, female nematodes in hairy roots were counted under the binocular.

FIG. 1 shows fluorescence microscopic images of 3-week old root clones from Nemata expressing the reporter GFP and the CRISPR-Cas9 cassette for knock-out of the Hs4 gene.

These images show expression of the GFP reporter gene, indicating presence of the knock-out cassette for Hs4.

FIG. 2 shows results from nematode resistance tests with four of the Nemata clones that were manipulated with the CRISPR-Cas9 knock-out cassette for the Hs4 gene. The number of females/cysts indicates susceptibility, and a low number of females/cysts indicates nematode resistance. The originally resistant control Nemata (NEMATA) and the susceptible beet (093161) were used as controls. The data show that knock-out of the Hs4 gene yielded significantly higher susceptibility, resp. abolished the resistance of the original NEMATA. FIG. 3 shows microscopic images of hairy roots infected with nematodes, A) of susceptible sugar beet line 93161 as a positive control, B) of Nemata roots in which the Hs4 gene has been knocked by CRISPR-Cas technology turning a resistant into a susceptible root. In FIG. 3, whitish beet cyst nematodes are indicated by arrows.

The susceptibility of the plants after specific inactivation of the Hs4 gene demonstrates that this gene confers resistance against nematodes.

Analysis of the originally nematode resistant sugar beet was by PCR, showing the presence of the Hs4 gene.

### Example 4: The Hs4 Gene Confers Nematode Resistance to Sugar Beets

As examples for species that is not related to Patellifolia procumbens, a non-resistant, i.e. nematode susceptible sugar beet was provided with a nucleic acid construct expressing Hs4.

The Hs4 encoding nucleic acid sequence was cloned under the control of the constitutive 35S promoter and transformed into roots of the susceptible sugar beet line. The plasmid containing the expression cassette for Hs4 has a nucleic acid sequence of SEQ ID NO: 13 (pBin35SRed).

As a reporter, the DsRed gene under the control of the CsVMV promoter was co-transformed.

For transformation the protocol as outlined in Example 3 was used. The primer-binding sites indicated can be used for designing complementary primers for PCR amplification of the intermediate nucleic acid section.

FIG. 4 shows fluorescence microscopic images of 12-d-old hairy roots from the susceptible beet line 093161, which was genetically manipulated to express the DsRed gene and carrying the Hs4-overexpression cassette from the pBin35SRed vector.

In total, 11 DsRed expressing roots were observed, and after infection with H. schachtii, different infection rates, corresponding to different expression rates of Hs4, were observed. It was found that resistance against nematodes correlated to expression of Hs4, with high expression of Hs4 conferring complete resistance, low expression of Hs4 yielding moderate resistance. The one clone that did not express Hs4 was highly susceptible.

FIG. 5 shows a graphic representation of these analytical results, giving expression levels of the Hs4 resistance gene product (left column) in relation to expression of the housekeeping gene GAPDH and number of female nematodes (right column) present on the hairy root clones (OEX1, OEX2, OEX3, OEX4, OEX5, OEX6, OEX7, OEX8, OEX9, OEX10), and the original susceptible control (093161).

As an example for a plant that is nematode resistant due to genetic manipulation to express the Hs4 gene as the only resistance gene, FIG. 6 shows a microscopic image of hairy roots of clone OEX7 in the presence of infecting nematodes. FIG. 6 confirms that the resistance conferred by expression of the Hs4 gene results in the absence of infection with the beet cyst nematode.

These data show that the Hs4 gene product confers resistance against nematodes to sugar beets.

### Example 5: The Hs4 Gene Confers Nematode Resistance Works into Plants of Different Genera

As an example for another plant genus, Brassicaceae, Arabidopsis thaliana, was transformed with an expression cassette encoding the Hs4 protein. The expression cassette contained the nucleic acid sequence encoding Hs4 under the control of the nematode inducible Hs1 promoter (nucleotides 9 . . . 1477 of SEQ ID NO: 15).

In short, the expression cassette was transformed into A. thaliana by the floral dip method. Seeds expressing the dsRed gene were selected. Plants were grown in the climate chamber and the T2 seeds were harvested. Two T2 populations were grown under sterile conditions and plants exhibiting the dsRed gene were inoculated with H. schachtii J2 larvae. The average number of females which had developed after 4 weeks was 1.4 and 14.7, respectively, while the control line Col0 exhibited 23.7 females on the average. This demonstrates that both populations are resistant to H. schachtii, but with varying degrees. One population exhibited almost complete resistance while resistance in the other population was moderate. This results typically reflects different integration sites and thus different expression intensities of the Hs4 gene. These results demonstrate that expression of Hs4 conferred complete resistance to this member of the Brassicaceae against nematode infection. The plasmid containing the expression cassette for Hs4 has a nucleic acid sequence of SEQ ID NO: 15 (hs1_hs4_pbin35sred).

1. A process for analysis of a plant in respect of resistance against nematodes, comprising analyzing nucleic acids of the plant for containing a nucleic acid sequence encoding a protein having a homology of at least 80% to SEQ ID NO: 1 and/or SEQ ID NO: 17.

2. The process according to claim 1, wherein the protein of SEQ ID NO: 1 is encoded by a nucleic acid sequence having a homology of at least 80% to SEQ ID NO: 3 or to SEQ ID NO: 2.

3. A resistance gene against plant parasitic nematodes encoding a protein having a homology of at least 80% to SEQ ID NO: 1 and/or SEQ ID NO: 17.

4. The resistance gene according to claim 3, the nucleic acid sequence encoding a protein having a homology of at least 80% to SEQ ID NO: 1 being arranged under the control of a promoter having SEQ ID NO: 4.

5. The resistance gene according to claim 3, wherein the protein is encoded by a nucleic acid sequence having a homology of at least 80% to SEQ ID NO: 3 or to SEQ ID NO: 2.

6. The resistance according to claim 3, within a plasmid vector suitable for gene transfer into plant cells.

7. The resistance gene according to claim 3 for use in protecting plants of the genera of Amaranthaceae, of Brassicaceae, of Poaceae, or of Solanaceae against nematodes.

8. A plant, genetically manipulated to contain a gene conferring resistance against plant parasitic nematodes, wherein the gene encodes a protein having a homology of at least 80% to SEQ ID NO: 1 and/or SEQ ID NO: 17.

9. The plant according to claim 8, wherein the gene encoding the protein having a homology of at least 80% to SEQ ID NO: 1 and/or SEQ ID NO: 17 is functionally arranged under the control of a promoter having SEQ ID NO: 4.

10. A plant genetically manipulated to contain a gene conferring resistance against plant parasitic nematodes, wherein the gene is under the control of a promoter having a homology of at least 80% to SEQ ID NO: 4.

11. The plant according to claim 10, wherein the gene conferring resistance against plant parasitic nematodes is comprised in a DNA portion inserted into the plant genome, which DNA portion consists of at maximum 3 kbp.

12. A process for obtaining a genetically manipulated plant, comprising crossing plants, one of which is a cultivar and the other one of which contains a resistance gene against plant parasitic nematodes encoding a protein having a homology of at least 80% to SEQ ID NO: 1, and selecting offspring plants by identifying offspring plants that contain the resistance gene against plant parasitic nematodes encoding a protein having a homology of at least 80% to SEQ ID NO: 1 and/or to SEQ ID NO: 17.

13. The process according to claim 1, wherein the offspring plant has essentially all traits of the cultivar and is genetically manipulated such that the resistance gene against plant parasitic nematodes is comprised in a DNA portion inserted into its genome, which DNA portion consists of at maximum 3 kbp.

14. A plant, genetically manipulated to comprise an expression cassette encoding a gene, characterized in that the expression cassette contains a promoter having a homology at least 80% to SEQ ID NO: 4, or the promoter comprising or consisting of SEQ ID NO: 4.
